(19) United States
(12) Patent Application Publication
(10) Pub. No.: US 2024/0008975 A1
(43) Pub. Date: Jan. 11, 2024

(54) DEVICE FOR RECEIVING AN INTRAOCULAR LENS AND METHOD FOR FOLDING AN INTRAOCULAR LENS

(71) Applicant: MEDICEL AG, Altenrhein (CH)

(72) Inventor: Volker Dockhorn, Altenrhein (CH)

(21) Appl. No.: 17/295,043

(22) PCT Filed: Nov. 19, 2019
(86) PCT No.: PCT/CH2019/050026

§ 371 Date: May 19, 2021

(30) Foreign Application Priority Data
Nov. 19, 2018 (CH) 01429/18

(51) Int. Cl. A61F 2/16

(52) U.S. Cl. CPC A61F 2/1691; A61F 2/167; A61F 2002/1681

(57) ABSTRACT

The invention relates to a device for receiving an intraocular lens with a lens body and at least one haptic, the device containing a first half-shell and a second half-shell which are each connected in an articulated manner to one another at a first of the longitudinal sides of the same by means of a first joint and can be moved relative to one another from an open position to a closed position, the inner surfaces of the half-shells forming a storage surface. In the open position, the half-shells form an open chamber for positioning or storing the lens in the relaxed state, and in the closed position, the half-shells form an enclosed chamber for positioning or storing the lenses in a folded state and for ejecting the lenses along the longitudinal extent of the half-shells.

### CROSS-REFERENCE TO RELATED APPLICATIONS

This application is a national phase entry under 35 U.S.C. § 371 of PCT/CH2019/050026 filed Nov. 19, 2019, which claims priority to Swiss Patent Application No. 01429/18 filed Nov. 19, 2018, the entirety of each of which is incorporated by the reference.

### TECHNICAL FIELD OF THE INVENTION

This invention relates to a device for receiving an intraocular lens and a method for folding an intraocular lens.

### BACKGROUND OF THE INVENTION

In cataract operations, artificial lenses, so-called intraocular lenses, are nowadays inserted into the capsular sac of the eye as a standard procedure.

During the operation, an ocular incision of typically 2 to 4 mm is made, through which the natural lens of the eye is first removed and then the implant is placed. For insertion, the artificial lens is inserted through the incision into the capsular sac in a folded state. As soon as the folded lens is inserted into the capsular sac, it unfolds back to its original shape.

The artificial lenses commonly used today consist of an optical lens body and, as a rule, two or more haptics projecting peripherally from the optical lens body at right angles to the optical axis of the lens body, which haptics serve as position springs for the lens body in the capsular sac. For example, there are two haptics which are arranged opposite one another on the lens body, each of which projects outward in a spiral manner in the same direction from the lens body. This haptic form, which is the most commonly used in the world, is known in the industry as open c-loop haptics.

Improved surgical tools and implants allow surgeons to make noticeably smaller incisions. Removal of the natural lens of the eye can nowadays be performed through incisions of less than 2 mm. However, this only makes sense if the intraocular lens can also be inserted through such a small incision.

Lens carriers or cartridges, in which a lens can be loaded and then ejected from the lens carrier by means of an injector have been developed in recent years for the insertion of an intraocular lens.

Examples of such lens carriers or cartridges and injectors are known, for example, from the patent specifications U.S. Pat. Nos. 6,267,768, 5,810,833, 6,283,975, 6,248,111, 4,681,102, 5,582,614, 5,499,987, 5,947,975, 6,355,046 and EP 1 290 990 B1, as well as the disclosures US 2004/0199174 A1, EP 1 905 386 A1 and WO 03/045285 A1.

In the injector device according to U.S. Pat. No. 4,681,102, the cartridge, which is formed as a folding device for the lens, and the injector nozzle, are separate parts. The cartridge can be inserted into the injector housing, whereupon the injector nozzle can be screwed onto the front of the injector housing.

In the injector device according to U.S. Pat. No. 5,582,614 and most of the previously known injector devices, such as U.S. Pat. Nos. 6,267,768, 5,810,833, 6,283,975 and 6,248,111, the cartridge exists as an integral unit with a folding device and an injector nozzle.

Intraocular lenses are supplied by the manufacturer in sterile packaging and, if necessary, in a liquid bath. Depending on the lens material, storage in a liquid may be necessary to protect the lens from dehydration. During the operation, the lens must be removed from the packaging in the sterile area and inserted or alternatively loaded into the loading device of an injector or directly into an injector with the cartridge provided. The lenses are very sensitive structures which can easily be damaged during transfer to a cartridge, during folding or during ejection from the injector nozzle. The risk of damage is particularly high for the haptics surrounding the optical part of the lens. In particular, when the lens is ejected from the injector, there is a risk that one of the two haptics will be pinched and consequently torn off, or that the front haptic will prematurely spread into the eye ahead of the lens body, which can be problematic, as shown below.

A common cartridge design, as shown by EP 1 290 990 B1, WO 03/045285 A1, EP 1 905 386 A1, U.S. Pat. Nos. 5,582,614 and 5,499,987, has two half-shells connected by a single hinge, whether or not with grooves or gripping means for clasping the edges of the lens.

For example, disclosure WO 03/045285 A1 shows a method for inserting an intraocular lens into the capsular sac of the eye, in which an overpressure is created to eject a lens floating in a lubricant from the injector nozzle. A compressible and deformable piston continuously adapts to the forward narrowing of the nozzle channel. The lens continues to fold as it travels and has a very small diameter at the end of its path. Because of the deformability of the piston, the end of the nozzle channel can be kept very narrow, consequently only a very small incision is required. A kit for performing the procedure contains a lens carrier and a lens. The lens is located in the lens carrier in a tension-free state. The lens and lens carrier may be carried by a holder and sterilely packed in a package until use, in the case in point a hydrophilic lens in a liquid that protects the lens from dehydration. During the operation, the lens holder together with the lens stored within is removed from the pack, inserted into the injector and folded. A lubricating fluid is then filled up through the channel. The lens can now be injected into the capsular sac of the eye to be treated.

In general, systems for prefolded lenses and systems for non-prefolded lenses can be distinguished in the prior art. In the systems without prefolded lenses, the lenses are only first folded during the ejection process for injection. An example of such a system without prefolded lenses is disclosed in U.S. Pat. No. 8,668,734 B2. In this disclosure, a cartridge consisting of two mold halves is used, into which the lens is inserted when the mold is open. This system also provides a recess laterally to the direction of ejection of the piston for the rear haptic, whereas the front haptic is mounted distally. There are, however, systems used in which the lens, when still unformed or alternatively unfolded, is loaded from the rear into a loading chamber (as shown, for example, in U.S. Pat. No. 5,810,833). The rear opening into which the lens is inserted is at least as wide and high as the lens itself. As long as a piston with a deformable tip (usually made of silicone or TPE) is used, then this piston tip must fill the entire volume of the rear opening so as to not run the risk of steamrollering the rear of the two haptics with this piston tip and thus getting it caught. The large volume of the deformable piston tip leads to high system forces when the piston is advanced into the maximum tapered area of the cartridge tip. The resulting forces can even be greater than the forces caused by the lens itself. In addition, the expandability or compressibility of the voluminous piston tip limits the minimum necessary inner diameter of the front nozzle tip. For this reason, systems in which the lenses are folded or alternatively prefolded for injection, prior to the ejection process, are still frequently used today. In particular, winged cartridges (as disclosed, for example, in U.S. Pat. Nos. 6,267,768, 6,248,111, 5,947,975 or U.S. Pat. No. 4,681,102) are used for this purpose, which always consist of at least two initially opened half-shells. The lens is prefolded when the at least two half-shells are closed and is thus present in the loading chamber in a prefolded state. The prefolding of the lens together with haptics by means of closing of the winged cartridge reduces the inner volume of the closed winged cartridge to almost half and thus allows the use of smaller deformable piston tips, which induces smaller incision forces for the same inner diameter of the cartridge tip or bring about the use of smaller cartridge inner diameters and thus smaller incisions for the same incision force. The disadvantage of these systems for prefolding the lenses is that the lens haptics in particular, and in the worst case even the optics, can become trapped between the two wings of the cartridge when they are closed. Trapped haptics usually tear off when the lens is pushed further forward, which is tantamount to a total loss of the lens.

A winged cartridge for receiving an intraocular lens in a loading chamber is presented In the disclosure WO 2015/070358 A2. The winged cartridge is formed by a first and a second half-shell, each half-shell having a wing handle on the longitudinal side. The two half-shells are connected in an articulated manner to one another at the respective wingless longitudinal side, by means of a first joint and can be moved relative to one another from an open position to a closed position, wherein the two half-shells form an ejection channel for an interocular lens in the closed position. Further, the disclosure describes that a front c-loop haptic is prefolded by the front haptic, which is blocked on the nozzle side by a stopper, being pressed against the optic by advancing the stamp and indirectly by advancing the lens. This prevents the front haptic from being injected into the eye in a stretched form. Initially, the haptic forms a bow, but the longer the haptic, the more this bow is compressed during further advancement in the nozzle. Depending on the haptic design, this can result in the haptic being more or less “folded” in the middle of its length, and in doing so the folded, but still lengthwise stretched haptic enters the eye. Due to this folding, the haptic can then still only have half its length, which can be considered disturbing by the operating surgeons with regard to possible capsular sac damage.

With hydrophilic lenses, there is also the problem that the front haptic, if it is only pressed against the optic on the nozzle side in a winged cartridge, unfolds very quickly in the eye, even before the optic itself enters the eye. In the worst case, this results in a haptic that is basically stretched when it enters the eye. The lens body following behind the haptic follows the movement of the haptic and can therefore rotate 180° when it enters the eye (upper side down and lower side up). This is very disturbing for the surgeon, since they must rotate the lens in the eye in tight quarters to correct the situation.

In both of the above situations, it would be desirable for the front haptic to not only be pressed up against the optic on the nozzle side, but rather to rest on the optic in such a way that the haptic is grasped or alternatively enclosed by the edges of the optic during folding or alternatively further folding of the optic and can only unfold when the optic unfolds in the eye after injection, thereby releasing the front haptic. Such folding is also called sandwich folding. It is the most typical form of haptic folding in all cartridges that function without pre-folding (and therefore without wings) and the folding occurs only and exclusively during ejection through the internal geometry of the cartridge. WO 2014/74860 A1 and EP 2′916′769 B1 describe a typical embodiment of such a cartridge without prefolding. The front haptic is placed on the optic by the operator (operating room nurse or surgeon) by means of the small slit at the rear end of the cartridge already when inserting the lens into the cartridge. This form of loading a lens by the operator, irrespective of the injector systems used, is referred to as a non-preloaded injector. Preloaded lenses, on the other hand, are those that have already been placed in the loading chamber of the injector by the lens manufacturer and sterilized along with it. A cartridge according to WO 2014/074860 A1 can only be used for non-preloaded lenses, since the lens including the haptic must already be stored in the loading chamber in a relaxed position and for the entire lifespan of the product, whereas WO 2014/074860 requires the active threading of the front haptic through the slot at the end of the cartridge by the end user (operating room nurse or surgeon). Similarly, the disclosure US 2009/0270876 also shows a closed cartridge to be loaded from the rear. Here too, an active operation and loading by the end user is required. A relaxed preloading is also not possible here.

With a prefolded system (i.e. with a winged cartridge), on the other hand, it has been extremely difficult up to now for both preloaded and non-preloaded lenses to place the haptics on the optics in such a way that sandwich folding can be achieved, i.e. the haptic is clamped in such a way that it can only first detach from the optic in the eye, this since during the prefolding step, both the optic and the haptic are brought into a U-shape and the front haptic thus does not reach the top of the lens optic in a controlled and reproducible manner even during further advancement of the lens, but rather continues to mostly still only be pressed against the optic. In addition, with winged cartridges, the risk of the front haptic becoming trapped between the wings when the loading chamber is closed increases dramatically, if the haptic has previously been successfully placed on the optic.

Disclosure 3562/CHE/2014 (Indian patent application under examination titled “Leading Haptic Positioner for Preloaded IOL Delivery System” by R. D. Thularsiraj) presents a method for sandwich folding starting from a preloaded system. This sandwich folding for a preloaded system works in such a way that the haptic is manually placed on the optic by means of a manually sliding hook or slider, which is inserted into the loading chamber through the nozzle tip. As soon as the wings of the loading chamber have been closed, the hook is pulled out again.

Disadvantages of this method are that at least two additional components are required, making the system more expensive, and for the physician, two additional steps are inevitably created by advancing the slider and later withdrawing it. Since preloaded systems are often compared with one another on the basis of the number of preparation steps, this together with the additional costs represents a competitive disadvantage.

Another disadvantage of this concept is that this approach is only used for fully preloaded lenses (usually used for hydrophobic lenses). However, it is not usable for so-called semi-preloaded lenses (usually used for hydrophilic lenses). In the latter case, the lens is indeed preloaded in the loading chamber, but at least the loading chamber is stored separately from the rest of the injector in liquid. Since the slider is located in the nozzle, the hook at the end of the slider cannot be positioned in contact with the separately stored lens in the loading chamber. When the loading chamber is inserted into the injector after removal from the storage liquid, the hook must be retracted through the slider to a point where it does not interfere with insertion of the loading chamber into the injector. Due to the limited size relationships, it is difficult to accomplish that a far-retracted hook will accurately grip and position the haptic. This problem does not exist with fully preloaded lenses, since the haptic is usually already applied (positioned) to the hook at the factory.

Another disadvantage of this concept is that the hook must be inserted through the nozzle, so the maximum size of the hook and slider is the size of the nozzle at its tip. For very small incisions, the inner diameter of a tip can be in the range from 1 mm to 1.5 mm, resulting in very delicate hook and slider geometries. The more delicate the system is designed, the more difficult it is to create a reliable system that reliably captures and positions the haptics.

### Purpose

The purpose of this invention is to provide an alternative device or system for preloaded intraocular lenses in which the front haptic does not protrude or alternatively project when ejected from the injector nozzle or alternatively injected into an eye. In particular, it would be desirable to place the front haptic of lenses, especially c-loop lenses, securely and reproducibly on the optics for injection into an eye, especially in such a way that the front haptic does not project or protrude during injection, but rather is clamped in the sandwich. A further purpose of this invention is to create an alternative device or system which, when injecting a lens into an eye, keeps the front haptic with the optic as long as possible, so that, as far as possible, the haptic unfolds from the optic only in the eye and this may be only when the optic unfolds. This purpose is to be solved in such a way that no additional parts, no additional costs and no additional application steps may be required. The system or alternatively the device shall be suitable for fully preloaded as well as semi-preloaded lenses. The system is also intended to function in conjunction with winged cartridges, in particular wing loading chambers which can be inserted into injector housings.

A further purpose of this invention is to provide a device for easy loading of an intraocular lens, which avoids the disadvantages of the described known systems and methods. It is further purpose of this invention to provide a device which folds an intraocular lens without damaging it during folding and/or injection. Furthermore, a device is to be provided which is optimized as regards the manipulation steps for the preparation of lens and injector. In particular, as few manipulation steps as possible should need to be carried out on the device after delivery of the lens and injector or alternatively immediately before the surgical intervention. In addition, a device should be provided which requires as few additional components as possible, in the best case, even without any additional components when compared to existing injectors, and thus fulfills the above-mentioned objectives without generating additional costs. Another goal is to create a device that requires only small incisions in the eye.

### SUMMARY OF THE INVENTION

According to the invention, the purpose is solved by a device, in particular a loading device, for receiving an intraocular lens, having a lens body and at least one haptic. The device comprises a first half-shell and a second half-shell, which are respectively connected in an articulated manner to one another at a first of their longitudinal sides by a first joint and can be moved relative to one another from an open position to a closed position, wherein the half-shells in the open position form an open chamber for positioning or storing the lens (esp. for positioning or storing the lens in the relaxed state), and in the closed position the half-shells form an enclosed (on the shell side) chamber (in particular a cylindrical chamber) for positioning or storing the lens (in particular for positioning or storing the lens in the folded state) and for ejecting the lens along the longitudinal extent of the half-shells. The device is advantageously characterized in that in at least one of the half-shells a recess is formed which is open at least from the inside of the half-shell and which is suitable to receive a front haptic of the lens in the closed position of the half-shells.

The invention has the advantage that the reservoir ensures that the front haptic of a preloaded lens with c-loop haptic is positioned on the optic in the loading chamber, in particular when using a loading chamber consisting of two half-shells connected to one another by a hinge, in such a secure and reproducible manner, in particular in the sandwich, that the haptic can only first unfold when the optic also unfolds in the eye. In particular, when a lens is injected into an eye, the front haptic cannot detach from the optic, i.e., the lens body, until the optic begins to unfold.

The advantageous embodiment features listed below, alone or in combination with one another, lead to further improvements of the device according to the invention and its application.

Advantageously, in the closed position of the half-shells, the recess forms an area, in particular a secondary space, which is arranged longitudinally to the enclosed chamber and is expediently configured such that the front haptic of the lens can be accommodated therein whereas the optics of the lens are positioned in the enclosed chamber.

Advantageously, each half-shell is equipped with at least one slide rail, the at least one slide rail being suitable for guiding the lens body and optionally the end of the rear haptic. As a result, the position of the lens body, i.e., the optics, in the device is more reliably predetermined. In particular, the sliding slide serves the purpose of guiding the ejection of the lens from the enclosed chamber or alternatively from an injector).

It is particularly advantageous that at least one of the half-shells, such as the second half-shell, is provided with a support for the front haptic, the support being suitable for guiding the front haptic, in particular the free end of the front haptic. During loading or for storage, the free end of the front haptic may be placed on the support in a substantially relaxed state. This support for guiding the front haptic may be placed parallel to the slide rail(s) for guiding the lens body, the slide rails may be located deeper in the open chamber than the support for guiding the front haptic. The support for the front haptic can be designed on the longitudinal edge of the second half-shell as an edge strip projecting into the open chamber. The support may be designed as a continuous guide structure arranged parallel to the slide rail(s). A front haptic resting on the support thus does not lie in a coplanar plane with the optics in the open chamber, but rather is elevated relative to the plane in which the optics lie. The support can alternatively be interrupted and/or, if necessary, be designed as a separate holder on a side of the device close to the nozzle. In particular, the support may be lowered on the side near the nozzle such that the front haptic, when resting on the support, is coplanar with respect to the optic.

Expediently, the support and the recess are configured and cooperate in such a manner that, upon closing of the half-shells from the open position to the closed position, the front haptic (43) extends increasingly beyond the support and out of the closed chamber that is formed, so as to come to rest in the recess when in the closed position.

Expediently, the chamber that is enclosed in the closed position of the half-shells substantially forms a channel, in particular a loading channel or alternatively an ejection channel. The enclosed chamber substantially forms a cylinder-like channel, the shell side of which is substantially defined by the half-shells, wherein the said recess forms an opening in the shell side. The recess for receiving the front haptic of the lens may form a lateral opening in the channel extending to the front side of the channel, in which the front haptic of the lens can be received.

Advantageously, wings are respectively arranged on a second longitudinal side of the two half-shells, in particular on the respective longitudinal edge of the half-shells, so that the half-shells can be moved relative to one another from an open position to a closed position by means of the wings and by rotation about the joint, wherein the recess may continue in at least one of the wings.

The recess may be such that the haptic is not substantially compressed or blocked when sliding into the recess during the closing of the half-shells. It is expedient that the recess is arranged on the inside of the wing and in such a way that, when the half-shells are in the closed position, the recess may be substantially adapted to the dimensions of the haptics.

It is further convenient that the recess is arranged such that it is formed in two substantially parallel surfaces (in particular, the inner surfaces of the two wings), wherein the said parallel surfaces (which may be substantially formed by the wings) have a reciprocal distance in the closed position of the half-shells which is at least equal to or exceeds the thickness (or diameter) of the haptic and optionally does not exceed five times or, optionally twice the thickness of the haptic. The recess between the substantially parallel surfaces is at least sufficiently wide and deep that the haptic is not substantially jammed, pinched or blocked when sliding into the recess located in the space spanning the width and the depth.

Optionally, a cover member may be pivotally disposed longitudinally on the first of the two half-shells, which cover member covers the open chamber when the half-shells are in the open position and is positioned substantially outside the enclosed chamber when the half-shells are in the closed position.

Optionally, a closure, in particular a snap closure, may be formed on the wings.

Optionally, a plug-in device for insertion into a receiving opening of an injector housing may be formed on one of the half-shells. Optionally, the plug-in device is provided on the second half-shell.

The device can be in one piece. From a manufacturing point of view, it is advantageous if the device is in one piece and may be made of plastic. Injection molding technology can be used in this case.

The device can expediently be designed as a cartridge for insertion into an injector, in particular into an injector housing. Advantageously, the device according to the invention may be an integral part of an injector.

Further disclosed herein is an injector having an injector housing and a plunger longitudinally displaceable in the injector housing for use with a device configured as a cartridge as described above.

Further disclosed is an injector having an injector housing with a loading device provided therein, a nozzle located upstream of the loading device, and a plunger longitudinally displaceable toward the nozzle in the injector housing, wherein the loading device is equipped with a chamber which can be pierced by means of the plunger for the purpose of ejecting a lens, wherein the loading device is designed as described above, in particular with collapsible half-shells. The injector is characterized in that, at least in the closed position of the half-shells, a folding edge is located or provided on the nozzle side of the half-shells, through which the front haptic is pressed or folded into the folded lens, in particular between the legs (or leg flaps) of the folded lens body, when the lens is pushed forward or folded in. The folding edge limits the recess toward the nozzle.

The folding edge may be provided between the loading device and the nozzle inlet of a nozzle, and, in particular, between the recess of the loading device and the nozzle inlet adjacent thereto. By way of example, the folding edge is formed at the nozzle inlet, expediently where the nozzle inlet of a nozzle abuts the loading device and in particular the recess of the loading device. Alternatively, the folding edge could be formed on at least one of the half-shells instead of on the nozzle inlet of the nozzle, or on the housing, so that it is formed between the loading device and the nozzle. In designs in which the nozzle and loading device are made in one piece, the folding edge is expediently placed between the nozzle area and the loading area.

It is expedient that in the closed position of the half-shells, the folding edge is positioned on the nozzle side in such a way that when the lens is pushed forward, the front haptic is folded or alternatively pressed into the folded lens, in particular between the legs of the folded lens body.

Further disclosed is a method of folding an intraocular lens comprising the steps of:
provision of a storage surface having a front, nozzle-near region and a rear, nozzle-far region, which are defined at least by a first half-shell and a second half-shell, the two half-shells being connected to one another in an articulated manner via a first joint,
application (in particular by placing or sliding on) of a lens onto the storage surface by bringing the lens body of the lens onto the storage surface so that a front haptic of the lens is positioned in the front region of the storage surface near the nozzle with respect to the lens body,
bringing the two half-shells together via the first joint by guiding the two half-shells toward one another by rotation about the first joint (i.e., in particular by folding the wings together), whereby the lens body is folded more or less in the middle so that a lens body which is initially substantially lenticular in its relaxed state is pressed into a shape with two legs folded toward one another. In accordance with the invention, the method is characterized in that, when the optical lens body is folded, the front haptic escapes from the space between the half-shells that close around the lens body, by means of the free end of the haptic slipping into a recess provided for this purpose in at least one of the half-shells.

Advantageously, the space created between the closing half-shells is substantially cylindrical, and the recess is created in such a way that the haptic can escape from the space on the side of the cylinder surface.

Advantageously, the lens is oriented on the storage surface in such a way that the front haptic of the lens comes to lie above the joint in such a way that the base of the haptic, which connects the haptic to the optical lens body, is positioned above the first half-shell and the end of the haptic is positioned above the second half-shell.

Advantageously, the lens can be inserted into the cavity in a relaxed state. This means that the lens can be inserted into the device (in particular by hand) without externally applied mechanical tension, in particular without bending or folding the lens.

It is expedient that the lens rests on the inner surfaces of the two half-shells, especially after manual insertion. This means that the lens rests at least on the inner surface of each half-shell at one point.

It is expedient that each half-shell is equipped with a support, e.g., designed as slide rails.

It is expedient that the optical lens body is enclosed at its edges by the two half-shells and is folded together with the half-shells, in particular in approximately the same direction.

In the process step involving bringing the two half-shells together, the process may include bringing them together until the two longitudinal edges of the two half-shells abut against one another (whereby a cover member—if present—is clamped).

According to the invention, the optics and optionally the rear haptics are lowered when the loading chamber is closed, whereas the front haptics are guided into the recess.

Further disclosed is a method for folding an intraocular lens and ejecting the lens through an injection nozzle, comprising the steps of:
folding the lens, such as according to the aforementioned method, whereby the lens body is folded more or less in the middle so that a lens body which is initially substantially lenticular in its unfolded state is pressed into a shape with two legs folded against one another,
pushing the folded optical lens body toward the injection nozzle, whereby the folded lens is increasingly compressed by an increasing constriction in the direction of the injection nozzle. The method for folding and ejection is characterized, in particular, in that during the push toward the injection nozzle, the front haptic that was initially positioned in the recess is pulled along and is clamped in a gap between the legs of the folded lens body, which gap narrows further as the injection moves forward. In doing so, the front haptic is expediently pulled out of the recess over an edge so that the front haptic is clamped between the legs of the folded lens body (41) (in particular, due to the pressure applied to it).

The device according to the invention can be used with relatively small incisions in the eye (in particular incisions with a diameter of less than 2.5 mm, optionally less than 2.2 mm, less than 2 mm, or less than 1.5 mm). This is possible because the device according to the invention has a loading chamber for a folded lens to be injected, which encloses the lens like a jacket and is thus closed (longitudinally). The lens is prefolded therein to a particularly small cross-sectional diameter and can be injected by means of a narrow cannula and through a—as described—particularly small incision. In a particularly advantageous manner, said device is used with a deformable stamp, in particular a silicone stamp (e.g., according to the disclosure document WO 03/045285 A1).

Additional advantages and objectives of this invention will be apparent from the following description.

### BRIEF DESCRIPTION OF THE FIGURES

Further embodiments of the invention result from the following description on the basis of the figures. The following figures, which are not true to scale, schematically show:

FIG. 1: an oblique view of the loading device according to the invention in the open position with the intraocular lens accommodated;

FIG. 2: a front view of the loading device according to the invention in the open position with the intraocular lens accommodated;

FIG. 3: a front view of the loading device according to the invention in closed position;

FIG. 4: a front view of the loading device according to the invention in closed position with the intraocular lens in place;

FIG. 5: an oblique view of the loading device according to the invention in closed position;

FIG. 6: an oblique view of an alternative loading device according to the invention in the open position with the intraocular lens accommodated;

FIG. 7: an oblique view of an injector with nozzle and inserted loading device according to the invention in closed position;

FIG. 8: an oblique view of an arrangement of the charging device according to the invention in closed position with the nozzle part in front (as contained in the arrangement in FIG. 7 in the injector with inserted charging device or integrated loading chamber);

FIG. 9: a longitudinal section through an arrangement of the charging device according to the invention in closed position with upstream nozzle part.

### DETAILED DESCRIPTION OF THE FIGURES

In the following, identical reference numbers stand for identical or

functionally identical elements (in different figures).

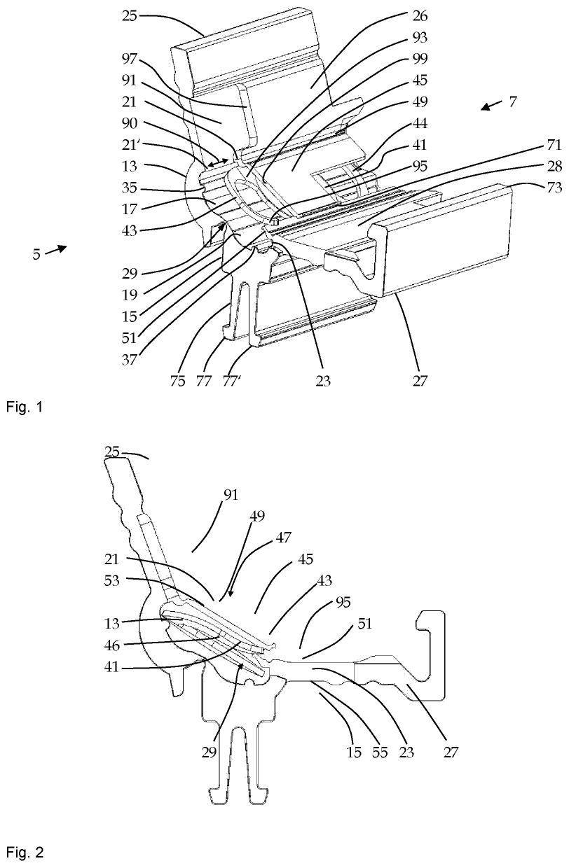

FIG. 1 to FIG. 5 show a device according to the invention in the form of a cartridge 3 which can be inserted into an injector housing 1. Alternatively, the device according to the invention can be part of an injector or be permanently integrated or formed in the injector.

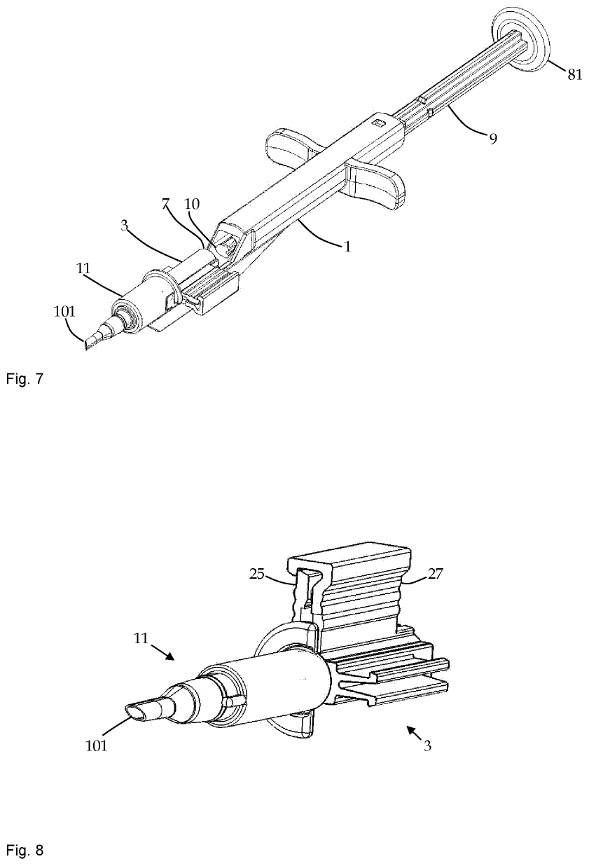

FIG. 7 shows an injector with cartridge 3 inserted.

FIG. 8 shows an arrangement of a cartridge 3 and a nozzle 11 as found in an injector.

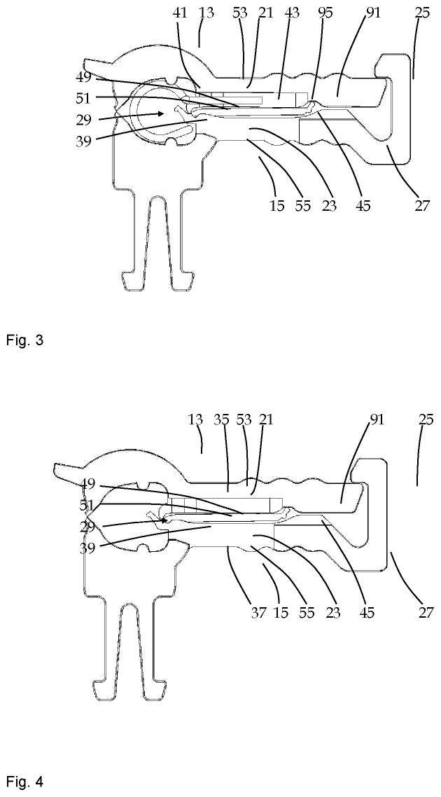

An injector is a surgical tool with a sleeve-like housing 1 and a plunger 9 (FIG. 7) received in the housing so as to be axially displaceable, wherein the plunger 9 can be pushed forward toward a nozzle 11 which is formed at the dorsal end of the injector housing 1 and is thus located in front of the plunger 9. An elastic stamp 10 is fitted to the plunger 9. A recess is provided in the jacket of the housing 1, into which a lens carrier, i.e., in particular the cartridge 3 described herein, can be loaded in such a way that the plunger 9 can be pushed through it during ejection. The cartridge 3 substantially connects behind the nozzle 11. Alternatively, an injector housing could be equipped with an integrated loading chamber, whereby the integrated loading chamber would be designed to be inserted into the housing in a manner substantially similar to the cartridge presented here. The lens carrier or cartridge 3 has a cylindrical loading channel 39 (FIG. 3 to FIG. 5), to which the injector nozzle 11 (distal end of the lens carrier), which tapers toward the tip, is axially connected (FIG. 8). The lens carrier or cartridge 3 is placed or inserted and held in the injector housing 1 in such a way that the plunger 9 is aligned with the loading channel. The plunger 9 can be pushed forward by manually pushing on the plunger end 81. When the plunger 9 is pushed forward, the plunger 9 enters the loading channel 39 and pushes out an injector stored therein through the injector nozzle 11 to, for example, be injected into an eye.

The cartridge 3 has a front end 5, i.e., located near the nozzle, and a rear end 7, i.e., located far from the nozzle (FIG. 1, FIG. 7, and FIG. 8). When inserted into an injector housing 1, the plunger 9 can be pushed from the rear end 7 into and through the cartridge 3, toward the front end 5 of the cartridge 3 and further into the injection nozzle 11.

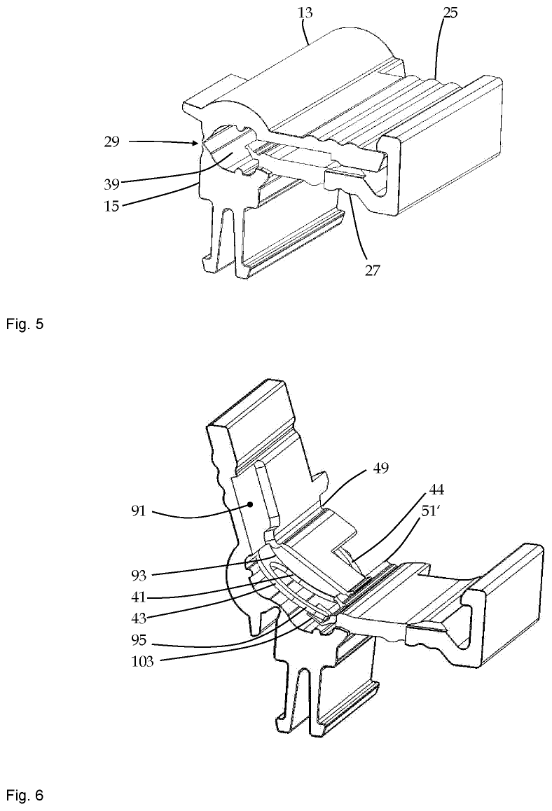

FIG. 1 shows a loading device according to the invention, which is advantageously designed as an insertable cartridge 3, which can be inserted into an injector, as shown in FIG. 7. The cartridge 3 includes two half-shells 13 and 15, which are connected in an articulated manner to one another via a first joint 29. The two half-shells 13 and 15 are, in particular, designed in the form of cylinder segments and are connected to one another in an articulated manner running longitudinally. The two articulated half-shells 13 and 15 together form a double half-shell. Each half-shell 13, 15 has an open half-channel on the inside with an inner surface 17 or alternatively 19. The inner surfaces 17 and 19 together form a storage surface. The storage surface 17, 19 is limited longitudinally by a first edge 21, which is implemented on the first half-shell 13, and a second edge 23, which is implemented on the second half-shell 15. Each half-shell 13, 15 has the edge 21 or alternatively the edge 23 on the side away from the first joint 29. Advantageously, a wing 25, 27 is arranged at each edge 21, 23. The half-shells 13 and 15 are connected to one another in an articulated manner in the longitudinal direction via the first joint 29, which is expediently designed, for example, as a hinge or film hinge. The two half-shells 13 and 15 are thereby lined up in such a way that the inner surfaces 17 and 19 of the two half-shells 13 and 15 lie next to one another, in particular adjoin one another laterally in the longitudinal direction (or merge into one another via the first joint 29) and form a common storage surface 17, 19. Longitudinally here means in alignment along the extension or in alignment of the half-channels. Due to the joint 29, the cartridge 3 can be shifted from an open position to a closed position. In the open position (FIG. 1 and FIG. 2), the two half-shells 13 and 15 form a kind of storage surface 17, 19 on which an intraocular lens can be placed and on which the lens can, if necessary, be stored without tension. When the cartridge 3 is closed, the longitudinal edges 21 and 23 of the two half-shells 13 and 15 approach one another and an intraocular lens resting on the storage surface 17, 19 is thereby grasped and folded. In the closed position (FIG. 3, FIG. 4, and FIG. 5), the two half-shells 13 and 15 together form a channel which is essentially closed on the lateral side, i.e., the aforementioned loading channel 39, which serves to hold the lens in the folded state ready for injection into an eye.

Intraocular lenses essentially consist of an optical lens body 41 (also called optics) and one or more haptics 43, 44, such as a first and a second haptic 43, 44 (FIG. 1), which in their most common embodiment protrude in the lens plane from the periphery of the lens body 41 in a spiral-like manner (in particular, in the same direction in a spiral-like manner) and are of flexible design. The haptic region connecting a haptic to the lens 41 may be referred to as a haptic attachment, e.g., 93. The free end 95 of the haptic may be referred to as the haptic end or haptic tip. In the open position of the half-shells 13, 15, the lens lies on the storage surface 17, 19 in such a way that the first haptic 43 (including the attachment 93 and tip 95) is positioned on the nozzle side with respect to the lens body 41 (referred to further as “front haptic”), whereas the second haptic 44 (referred to further as “rear haptic”) is positioned nozzle-far.

To accommodate the front haptic 43, the edge 21 extending longitudinally of the half-shell 13 is offset or downsized in its longitudinal extent in a partial area 21′ or, in other words, forms an offset so that, when the half-shells 13, 15 are in the closed position, the inner surface 17 of the first half-shell 13 has a breach (or opening) 90 in the channel wall defined by the edges 21, 21′ and 23. The offset may be continued into the wing surface, for example such that the wing thickness is reduced below the offset. Thus, in the closed position of the half-shells 13, 15, i.e. when the edges 21 and 23 or the wings 25 and 27 are closest to one another or, as the case may be, substantially in contact with one another, there is a recess 91 at the side of the loading channel 39 which extends from the edges 21′, 23 between the wings 25, 27. The breach 90 or alternatively the recess 91 are configured to allow the front haptic 43 to escape from the loading channel 39 that is being formed when the lens body 41 is folded. The said offset and thereby the breach 90 or the recess 91 that results in the closed position are expediently arranged on the nozzle side or at least near the nozzle side (i.e., in the front part of the cartridge 3), so that the front haptic 43 can escape from the channel space 39 into the recess 91 through the breach 90 formed by the offset edge 21′ (gap between edge 21′ and 23) when the optics are folded.

The breach 90 or the recess 91 is expediently accessible at least from the inside of the half-shell 17, 19, in particular from the enclosed chamber 39, whereby the recess is suitable for receiving a front haptic 43 of the lens in the closed position of the half-shells 13, 15. Whereas the lens 41 is positioned folded in the chamber 39 after closure of the wings 25, 27 (i.e., in closed position), the front haptic 43 is positioned in the recess 91.

To the extent that wings 25, 27 adjoin edges 21, 23, recess 91 may continue from edge 21 into the wing 25. In the practical embodiment, the wing 25 is provided with a smaller wall thickness in a partial area, so that the recess 91 results on the inner surface 26 of the wing 25.

Optionally, the recess 91 is open not only toward the half-shell inner side 17 but also toward the nozzle side.

Although in the illustrated embodiment example the breach 90 or the recess 91 is created in edge 21 and wing 25 of the first half-shell 13, alternatively or additionally a corresponding recess could be created in edge 23 and wing 27. In a further alternative embodiment, a functionally similar recess elsewhere in a half-shell 13 or 15, for example as a continuous hole (not shown), could expediently be applied in the half of the cartridge 3 closer to the nozzle, and/or close to the edge 21 or 23.

When the injector is loaded, the relaxed interocular lens, in particular its optics 41, rests expediently between the longitudinal edges 21, 23, and/or on a guide structure of the storage surface 17, 19, the guide structure consisting here, for example, of slide rails 35, 37 which are formed longitudinally on the storage surface 17, 19. The slide rails 35, 37 are designed in particular as ribs.

The two longitudinal edges 21, 23 are suitably fitted with longitudinally oriented strips 49, 51 (strip 51 is also referred to as guide rail in the following). The strips 49, 51 are both suitably transversely curved to the longitudinal direction and, if necessary, have tapered longitudinal sides. The curvature of the strips 49, 51 is such that, when the cartridge is closed, the two strips complement one another to form a semicircular bulge which extends into the closed channel 39. In lateral continuation of the inner surfaces 17, 19 at the respective longitudinal edge 21, 23, the strips 49, 51 form a mating surface to the respective inner surface 17, 19, whereby a kind of internal groove 53, 55 is formed on both sides at the respective longitudinal edge 21, 23 (FIG. 3). Strips 49, 51 or, in particular, the grooves 53, 55 defined thereby (and optionally the slide rails 35, 37) can cooperate as a guide system for inserting and folding a lens, in particular the lens body 41. The strips 49, 51 can be continuous in the longitudinal direction (as shown in FIG. 1) or interrupted (FIG. 6). The guide system is expediently set up parallel to the longitudinal extent of the half-shells 13, 15. The strip 51 on the second half-shell 15 projects sufficiently far into the half-shell 15 that the tip 95 of the front haptic 43 can be placed on the strip 51, whereas at the same time the lens body rests on both sides under the strips 49, 51 on the inner surfaces 17, 19, in particular on the slide rails 35, 37.

The procedure for loading the lens in the device or alternatively loading chamber according to the invention is, for example, as follows: the lens is loaded into the loading chamber by advancing the optics 41 under the strip or guide rail 51 into the preloaded position. During this process, the haptics can already be somewhat prefolded, under slight tension. The haptics are prefolded in the direction of the optics. However, they can also be preloaded in relaxed state and prefolded in the further course of loading by the end user via the silicone stamp 10 of the piston. The front haptic 43 is placed on the guide rail 51, which is designed, in particular deep enough, so that the haptic cannot fall down on its own, in contrast to common loading chambers (such as in WO 2015/070358 A2). For example, the guide rail 51 is continuous and extends substantially in a straight line, so that the haptic 43 is raised slightly above the plane of extension of the lens body 41 by being lifted onto the guide rail 51.

If, alternatively, the front haptic 43 was to come to lie in the same plane as the optic 41, then the guide rail in a front area 103 (i.e., on the nozzle side) lies lower than in the rear area (i.e., on the plunger side) or alternatively is correspondingly located lower. The area 103 can optionally be designed as a separate shelf, which is in particular separated from a strip 51′ and, if necessary, offset from the longitudinal axis of the strip.

FIG. 6 shows an alternative loading device in which an area 103 of this type for the front haptic 43 is set lower on the nozzle side than the supporting surface defined by the strip 51 in FIG. 1. This has the advantage that the front haptic 43 and the optic 41 are mounted in the same plane, in this respect the sliding rail 37, for the optic 41, and the separate supporting surface 103, for the front haptic 43, are correspondingly designed to match one another. Depending on the lens material, this can be particularly advantageous for long-term storage.

For example, whereas the inner surfaces 17, 19, in particular the slide rails 35, 37, serve as supporting surfaces for the optics 41 in the relaxed state (FIG. 1), the strip 51 can serve as a supporting surface for the front haptic 43 in the relaxed state. Upon closing of the loading chamber, the ledges 49 and 51 press on the optics and rear haptics lying below and cause the optics and rear haptics to fold downward (i.e., to deflect toward the first joint 29, see FIG. 3), whereas the front haptic 43, which rests on the ledge 51, stretches through the lateral breach 90 or into the lateral recess 91 in the loading channel that is being formed.

The supporting surface for the front haptic 43 on the strip 51 of the second half-shell 15 and the edge offset 21′, which defines the breach 90 or alternatively the recess 91 of the first half-shell 13, are matched to one another in such a way that, when the two half-shells 13, 15 are closed to form the closed channel 39, the front haptic 43 passes over the strip 51 (or, according to the alternative embodiment, over the separate supporting surface 103) into the breach 90 and thus the recess 91.

The strip 51 shown here has a multifunctional purpose. Like the strip 49, it serves together with the latter as a guide structure for inserting the lens and as a folding aid in that, when the wings 25, 27 are closed, they force the optic 41, which lies under the strips 49, 51, to bend downwards toward the first joint 29. In addition, the ledge 51 serves as a guide for the front haptic 43, firstly during insertion of the lens into the loading device, whereby the front end of the front haptic 43 is inserted on the ledge in the longitudinal direction thereof, and secondly during closing, whereby the front haptic is guided transversely across the ledge 51 out of the closing chamber 36.

Previously, it was important that the haptics did not come to rest on the guide rail, since they would then become trapped between the wings during closing and tear off when the lenses were pushed forward. According to this disclosure, however, it is intended that the front haptic 43 rest on the guide rail 51. When the wings 25, 27 are closed, the lens or alternatively its body 41 folds into a “U” shape. The front haptic 43 can thereby slide between the wings 25, 27, where due to the recess 91 a cavity is created in which the front haptic 43 comes to freely rest (i.e., unclamped). When the front haptic 43 lies freely, it is pulled back as soon as the optic 41 is pushed forward. In doing so, it is pulled lengthwise out of the cavity into the optic 41, which is folded in a U-shape but still open at the top (hence the “U” shape). The further the lens is pushed forward, out of the loading chamber in the direction of the nozzle 11, the more the “U” closes in the narrowing passage and the front haptic 43 is enclosed between the legs of the U-shaped folded optic 41.

As shown in FIG. 1, the recess 91 (i.e., the breach 90 or alternatively the breach 90 with recess 91) is formed on the one half-shell (here, on the first half-shell 13), whereas the ledge 51 is formed on a corresponding region (i.e., in a corresponding region overlapping in longitudinal or alternatively axial extent of the loading device) of the other half-shell (here, the second half-shell 15). As a result, a region of the ledge 51 (here, in particular, the front or substantially nozzle-sided region of the ledge 51) is opposite the breach 90, which forms the opening on the chamber side toward the recess 91.

After the lens has exited into the eye, the previously folded optic 41 opens and releases the haptic 43.

The folding process described here with controlled clamping of the front haptic 43 in the folded optic 41 results in a delayed release of the front haptic 43 during unfolding when compared to the folding process in conventional devices with folding or wing chambers, so that the front haptic 43 is only released in the eye when the optic 41 unfolds. Due to the provision of a loading chamber with a breach 90 in the wall 17 of the loading channel 39 and a recess 91 extending from the breach 90 along the length of the loading channel 39, the entire folding process (folding and unfolding) is modified, in particular without the need for further components.

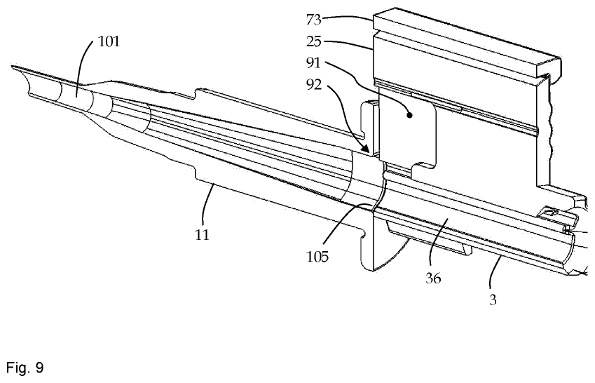

FIG. 8 shows an arrangement of the cartridge 3 with a directly upstream nozzle 11. FIG. 9 shows a longitudinal section through such an arrangement. In the longitudinal section, the loading channel 36 of the cartridge 3 can be seen, the loading channel to which the nozzle channel 101 is connected. Loading channel 36 and nozzle channel 101 form a passage for the lens, which is pre-folded in the loading channel and successively folded and pressed more strongly in the narrowing nozzle channel 101 as it passes through from nozzle inlet 105 to nozzle outlet 107. The recess 91 is located to the side of the loading channel 36. The transition from the loading channel 36 to the nozzle channel 101 has an edge 92 in the area of the recess 91. The edge 92 forms a spatial boundary between the recess 91 and the nozzle channel 101. In particular, the nozzle inlet 105 (i.e., the inlet to the nozzle channel) forms a boundary edge 92 in the region of the recess 91, over which the front haptic of the lens, stretched into the recess 91, is drawn when a lens is pushed forward. The edge 92 is expediently aligned in a manner substantially transverse to the longitudinal extent of the half-shells or transverse to the injection or alternatively ejection direction. By means of this edge 92, the front haptic 43 is pressed and folded into the still partially open “u” or between the lens legs of the folded lens when the lens is pushed forward (in particular, when it is pushed forward from the loading chamber 39 toward the nozzle 11), which leads to sandwich folding when the increasingly folded lens is pushed further forward. The edge 92 between the nozzle channel 101 and the recess 91 can therefore also be referred to as the folding edge.

A closure 73, in particular a snap closure, is formed on the wings 25, 27.

A plug-in device 75 is formed on the half-shell 15. This plug-in device 75 is used for insertion into an opening of an injector housing 1. Struts 77, 77′ with barbs, for example, serve as insertion means. This creates a solid connection between these parts after the cartridge 3 has been inserted into the injector housing 1.

The injector shown in FIG. 7 substantially consists of the injector housing 1 and the plunger 9, which is displaceable in the housing, for transporting and ejecting the lens. In the embodiment shown, the cartridge 3 and, as the case may be, the injector nozzle 11 represent separate parts that can be inserted into the injector housing 1, but they can also be designed as one part. The plunger 9 is mounted in an initial position in the injector housing, and locked, so that it is not in the way when the cartridge 3 is inserted. By actuating the piston at the piston end 81, an inserted lens can be ejected by pushing against the plunger 9.

In summary, the following can be stated:

A device for folding an intraocular lens has two half-shells 13, 15 connected by a hinge 29, which can be moved relative to one another and closed against one another, e.g. by means of wings 25, 27 formed on the half-shells 13, 15, in order to fold an inserted intraocular lens and at the same time keep it ready for ejection in a loading carrier channel which is formed by closing the half-shells 13, 15. Between the edges 21, 23 of the half-shells 13, 15, which are closed against one another, a recess 91 is formed on the nozzle side, into which the front haptic 43 deviates when the optic 41 is folded. The edge 92, which is located on the nozzle side in front of the recess 91 and is designed in such a way that when the lens is ejected from the loading chamber, the front haptic 43 is pulled out of the recess 91 and over the edge 92 and, due to the resulting pressure on the front haptic 43, the haptic 43 is folded in the sandwich between the folded but upwardly open (toward the recess) optics legs. As a result, it is now possible to also bring preloaded lenses into a sandwich fold making use of wing cartridges (then, in particular, by folding together of wings) or more generally of hinged cartridges for the purpose of injection into an eye, which results, upon injection through the nozzle, in the unfolding of the haptics only taking place with the unfolding of the optics (i.e., not before the unfolding of the optics). This device also enables the aforementioned form of front haptic folding, even in the case of a loading chamber that is separate from the rest of the injector.

In order to avoid a clamping of the lens and in particular of its haptics 43, 44 when closing the half-shells 13, 15, the cartridge 3 can be equipped with a cover member 45, as detailed in WO 2015/0730358 A2. A cover member pivotally arranged on the longitudinal wing side of the first of the two half-shells serves to cover an interocular lens which is inserted between the half-shells in the open position of the half-shells. The cover member thereby longitudinally covers an open chamber formed by the half-shells and simultaneously protects the lens or alternatively holds it in position. Upon closing of the two half-shells, the cover member slides over the edge of the second half-shell, so that as soon as the two half-shells are in the closed position, the cover member is positioned substantially outside the chamber defined by the two half-shells. The cover member is (and remains) attached to the first half-shell in both the open position and the closed position. This system has the advantage of reducing the risk of the lens haptic becoming trapped during folding.

The cover member 45 is movably arranged or alternatively fastened to the longitudinal edge 21 of the first half-shell 13, in particular in a movable or foldable manner (similar to a single-leaf swing door). The cover member 45 is advantageously designed as a lid plate, in particular as a flat, dimensionally stable lid plate. In an open position of the cartridge 3, the cover member 45 spans the storage surface 17, 19 from the longitudinal edge 21 of the first half-shell 13 to the longitudinal edge 23 of the second half-shell 15.

The cover member 45 is advantageously movably fixed to the longitudinal edge 21 of the first half-shell 13 via a second joint 47. Expediently, the joint 47 is a hinge, in particular a film hinge, and is configured as a bending groove or folding region.

The axes of rotation of the first and second joints 29 and 47 are aligned parallel to one another.

The cover member 45 closes toward the longitudinal edge 23 due to gravity and/or spring tension in the second joint 47. Since the cover member 45 is structurally self-supporting (i.e., sufficiently rigid), in the open position of the cartridge 3 a covered chamber 46 is formed between the first half-shell 13, the second half-shell 15 and the cover member 45. The second joint 47 is arranged on a strip 49 projecting from the longitudinal edge 21 of the first half-shell 13. On the opposite side, i.e., on the longitudinal edge 23 of the second half-shell 15, a second protruding strip 51 is formed, which serves as a support for the cover member 45 when the cartridge 3 is open. The tapering longitudinal side of the strip 49 expediently merges into a film hinge.

The strip 49 is formed with a concave bending groove on the outside of the chamber. In particular, the bending groove has a linear material displacement in the longitudinal direction of the first half-shell 13, whereby the ability to bend of the material is produced. This allows the cover member 45 to be folded toward the first wing 25 in an articulated manner. In particular, the bending groove functions as a film hinge.

The longitudinal edge 23, i.e., in particular the strip 51, on the second half-shell 15 and the free end 58 of the cover member 45 are configured in such a way that when the two wings 25, 27 are pressed together (i.e., when the wing handles are brought together by hand), the cover member 45 or its free end 58 are pushed forward and slide along the wing surface 28. The strip 51 can thus form a kind of cover member add-on.

In the closed position of the cartridge 3 (FIG. 3), the cover member substantially lies outside the enclosed chamber 39 formed by the chamber 46 and between the wings 25, 27.

During the closing process of the cartridge 3, in which the wings 25, 27 of the cartridge 3 are brought together, the cover member 45 slides from its latching position at the edge 23 onto the inner wing surface 28 of the second wing 27 and along this wing surface 28 out of the cavity 46 being closed or alternatively out of the cavity 39 being formed.

With respect to the cover member, the device is, in particular, characterized by the following features:
a cover member 45 is pivotably arranged longitudinally on the first of the two half-shells 13, which cover member in the open position covers the open chamber 46 and in the closed position is positioned substantially outside the enclosed chamber 39.
the cover member has, on the nozzle side, an edge development 99 which is set back with respect to a nozzle sided end face of the half-shells 13, 15, set back in such a way that, when the lens is inserted, the front haptic of the lens is not covered by the cover member, whereas the optics of the lens are covered by the cover member.
the cover member 45 is set back relative to a nozzle sided end face of the half-shells 13, 15 to such an extent that, when a lens is inserted, the front haptic 43 is not covered by the cover member, whereas the optic 41 is covered.
the cover member 45 is plate-shaped.
the cover member 45 is pivotably arranged on the longitudinal edge 21 of the first half-shell 13.
the cover member 45 is connected to the first half-shell 13 via a second joint 47, which is designed, for example, as a hinge, in particular as a film hinge.
a bending groove is formed on the cover member surface deviating from the inner surface 17 of the first half-shell 13.
a cover member add-on 51 is formed on the longitudinal edge 23 of the second half-shell 15.
upon closing of the two half-shells 13, 15, the cover member 45 slides over the longitudinal edge 23 of the second half-shell 15 out of the closing chamber 46.
in the closed position of the half-shells 13, 15, the cover member 45 is positioned substantially outside the enclosed chamber 39 between the wing handles 25, 27.
the recess 91 on the first wing 25 has an edge development 97 which, in the closed position, essentially follows the nozzle sided edge development 99 of the cover member 45 or (in comparison with the nozzle sided edge development 99 of the cover member 45) is set back further from the nozzle sided end face of the half-shells 13, 15.
in the closed position, the cover member 45 substantially lies outside the enclosed chamber 39 and between the wings 25, 27.
that the cover member 45 is designed as structurally self-supporting.
in the process step for bringing together the two half-shells 13, 15, the cover member 45—if present—is clamped until the two longitudinal edges 21, 23 of the two half-shells 13, 15 mutually come into contact against each other.

Whereas specific embodiments have been described above, it is obvious that different combinations of the embodiment possibilities shown can be used, insofar as the embodiment possibilities are not mutually exclusive.

Whereas the invention has been described above with reference to specific embodiments, it is apparent that changes, modifications, variations and combinations can be made without departing from the spirit of the invention.

1. A loading device for receiving and folding an intraocular lens with a lens body and at least one haptic, comprising:
a first half-shell having a first inner surface and a second half-shell which are having a second inner surface, the first and second half-shells connected in an articulated manner to one another at a first longitudinal side of the first half-shell and the second half shell by a first joint, the first half-shell and the second half shell configured to be movable relative to one another from an open position to a closed position, the first and second inner surfaces of the first and second half-shells forming a storage surface, wherein
in the open position, the first and second half-shells form an open chamber for positioning or storing the lens in a relaxed state, and
in the closed position, the first and second half-shells form an enclosed chamber for positioning or storing the lens in a folded state and for ejecting the lens along a longitudinal extent of the first and second half-shells, whereby a recess is formed in at least one of the first and second half-shells, which recess is open at least from an inside of the at least one of the first and second half-shell and is configured for receiving a front haptics of the lens in the closed position of the first and second half-shells.

2. The loading device of claim 1, wherein in the closed position of the first and second half-shells, the recess forms a secondary space arranged longitudinally to the enclosed chamber and is configured so that the front haptic of the lens can be accommodated therein and the optics of the lens are positioned in the enclosed chamber.

3. The loading device of claim 1, wherein at least one of the first and second half-shells is provided with a support for the front haptic, the support configured for guiding a free end of the front haptic.

4. The loading device of claim 3, wherein the support and the recess are configured and cooperate in such a way that upon closing of the first and second half-shells (17, 19), from the open position to the closed position, the front haptic increasingly extends beyond the support and out of the forming closed chamber to come to rest in the recess in the closed position.

5. The loading device of claim 1, further comprising wings respectively arranged on a second longitudinal side of the first and second half-shells, so that the first and second half-shells can be moved relative to one another from the open position to the closed position by the wings and by rotation about the joint, wherein the recess continues at least in one of the wings.

6. The loading device of claim 1, wherein each half-shell is provided with at least one slide rail, wherein the at least one slide rail is configured for guiding the lens body and optionally an end of a rear haptic.

7. The loading device of claim 1, further comprising a cover member pivotably arranged longitudinally to the first half-shell, the cover member, in an open position, covers the open chamber, and in a closed position, is positioned substantially outside the enclosed chamber.

8. The loading device of claim 1, wherein the loading device comprises a cartridge for insertion into an injector or an injector housing.

9. An injector, comprising:
an injector housing with a loading device comprising:
a first half-shell having a first inner surface and a second half-shell having a second inner surface, the first and second half-shells connected in an articulated manner to one another at a first longitudinal side of the first half-shell and the second half shell by a first joint, the first half-shell and the second half shell configured to be movable relative to one another from an open position to a closed position, the first and second inner surfaces of the first and second half-shells forming a storage surface;
wherein in the open position, the first and second half-shells form an open chamber for positioning or storing the lens in a relaxed state; and
wherein in the closed position, the first and second half-shells form an enclosed chamber for positioning or storing the lens in a folded state and for ejecting the lens along a longitudinal extent of the first and second half-shells;
whereby a recess is formed in at least one of the first and second half-shells, which recess is open at least from an inside of the at least one of the first and second half-shells and is configured for receiving a front haptic of the lens in the closed position of the first and second half-shells,
a nozzle mounted upstream of the loading device; and
a plunger longitudinally displaceable in the injector housing toward the nozzle, wherein the loading device is equipped with a chamber that can be pushed through by the plunger for ejecting a lens,
wherein at least in the closed position of the first and second half-shells, an edge is arranged on a nozzle side of the half-shells, by which an edge of a front haptic is pressed or alternatively folded into a folded lens between legs of a folded lens body when the lens is pushed forward.

10. The injector of claim 9, wherein the edge delimits the recess toward the nozzle, and is formed at a nozzle inlet.

11. A method of folding an intraocular lens comprising:
using a loading device having a storage surface having a front, nozzle-near region and a rear, nozzle-far region defined by at least a first half-shell and a second half-shell, the two half-shells connected in an articulated manner with one another by a first joint;
placing a lens on the storage surface, by bringing a lens body of the lens onto the storage surface, such that, with respect to the lens body, a front haptic of the lens is positioned in the front region of the storage surface near the nozzle; and
bringing the two half-shells together via the first joint, by guiding the two half-shells toward one another by rotation about the first joint, whereby the lens body is folded approximately in a center so that the lens body that was initially substantially lenticular in a relaxed state is pressed into a shape with two legs folded toward one another,
wherein during folding of the optical lens body, the front haptic escapes from a space between the half-shells that close around the lens body by a free end of the haptic slipping into a recess provided in at least one of the half-shells.

12. The method of claim 11, wherein the space formed between the closing half-shells is cylinder-shaped, and the recess is formed in such a way that the haptic can escape from the space on a cylinder surface side.

13. The method of claim 11, wherein the lens is oriented on the storage surface in such a way that the front haptic of the lens comes to be situated over the joint such that a haptic attachment is positioned over the first half-shell and an end of the haptic is positioned over the second half-shell.

14. The method of claim 11, wherein the lens is inserted into a cavity in an un-tensioned state.

15. The method of claim 11, wherein the optical lens body is enclosed at its edges by the two half-shells and is folded together with the half-shells in approximately a same direction.

16. The method of claim 11, wherein upon closing of a loading chamber, an optic and rear haptic lower whereas a front haptic is guided into the recess.

17. A method for folding an intraocular lens and ejecting the lens through an injection nozzle, comprising:
folding the lens having a lens body, whereby the lens body is folded approximately in a center such that the lens body which was initially substantially lenticular in a relaxed state is pressed into a shape with two legs folded toward one another;
pushing the folded optical lens body toward the injection nozzle, wherein the folded lens is increasingly compressed by an increasing constriction in a direction toward the injection nozzle;
and
during a push toward the injection nozzle, pulling along a front haptic that was initially positioned in a recess and clamping the front haptic in a gap between the legs of the folded lens body, which gap narrows further as injection moves forward.

18. The method of claim 17, wherein the front haptic is pulled along and drawn out of the recess over an edge, whereby the front haptic is clamped into a gap between the legs of the folded lens body that continues to narrow as the lens is pushed forward.
